(19) United States
(12) Patent Application Publication
(10) Pub. No.: US 2026/0061112 A1
(43) Pub. Date: Mar. 5, 2026

(54) AURILAVE AND VISUAL AURILAVE

(71) Applicant: WenMing Li, Shenzhen City (CN)

(72) Inventor: WenMing Li, Shenzhen City (CN)

(21) Appl. No.: 19/380,780

(22) Filed: Nov. 5, 2025

Related U.S. Application Data
(63) Continuation-in-part of application No. 18/078,126, filed on Dec. 9, 2022.

(51) Int. Cl. A61M 3/02

(52) U.S. Cl. CPC A61M 3/0258; A61M 3/0287

(57) ABSTRACT

The present invention discloses an aurilave, including: a water storage tank provided with a mounting port; a housing detachably arranged on the mounting port of the water storage tank; and an ear-washing assembly including a control plate arranged in the housing, a water-pumping motor arranged in the housing and electrically connected to the control plate, a water inlet pipe connected to a water inlet port of the water-pumping motor at one end thereof, a water outlet pipe connected to a water outlet port of the water-pumping motor at one end thereof, a nozzle arranged on the water outlet port of the water outlet pipe, and a transparent water baffle arranged on the water outlet pipe.

### CROSS REFERENCE OF RELATED APPLICATION

This is a Continuation-In-Part of application Ser. No. 18/078,126 filed Dec. 9, 2022, wherein the entire content of which is expressly incorporated herein by reference.

### BACKGROUND OF THE PRESENT INVENTION

### Field of Invention

The present invention relates to the technical field of an ear-cleaning apparatus, and particularly to an aurilave and a visual aurilave.

### Description of Related Arts

In people's daily ear-canal cleaning, forceps, an ear spoon and other hard objects are commonly used for cleaning an ear canal. Since a user cannot see a situation in the ear canal during self-cleaning of the ear canal, and it is difficult to control a force during use, it is easy to damage an ear membrane or the ear canal during the ear-canal cleaning, which leads to an ear disease. A safety risk exists in a method for cleaning the ear canal in the prior art.

### SUMMARY OF THE PRESENT INVENTION

A main objective of the present invention is to propose an aurilave, which is intended to improve safety of ear cleaning.

To achieve the above objective, the aurilave proposed according to the present invention includes:
a water storage tank provided with a mounting port;
a housing detachably arranged on the mounting port of the water storage tank; and,
an ear-washing assembly including a control plate arranged in the housing, a water-pumping motor arranged in the housing and electrically connected to the control plate, a water inlet pipe connected to a water inlet of the water-pumping motor at one end thereof, a water outlet pipe connected to a water outlet of the water-pumping motor at one end thereof, a nozzle arranged on the other end of the water outlet pipe, and a transparent water baffle arranged on the water outlet pipe, the other end of the water inlet pipe extending into the water storage tank.

Optionally, the ear-washing assembly includes a sprinkler seat. One end of the sprinkler seat is fixed in the housing and communicated with the water outlet pipe, and the other end of the sprinkler seat protrudes out of the housing. The nozzle is arranged at one end of the sprinkler seat located outside the housing.

Optionally, the ear-washing assembly further includes a front cover arranged on the housing and an LED lamp plate arranged within the front cover and emitting light in a direction toward the nozzle. A middle of the front cover is opened and provided with a fixing hole for fixing the sprinkler seat. The front cover is opened and provided with a light-transmitting hole for accommodating lamp beads of the LED lamp plate.

Optionally, the front cover is formed with a sealing groove relative to the other side of the LED lamp plate. The light-transmitting hole is located at a bottom of the sealing groove. The aurilave further includes a transparent sealing ring arranged within the sealing groove.

Optionally, the transparent water baffle is in a form of a disk raised in a middle thereof.

Optionally, the ear-washing assembly further includes a rear cover and a power switch arranged on the rear cover, and the power switch is electrically connected to the control plate.

Optionally, the ear-washing assembly further includes a key switch arranged on the housing for controlling the water-pumping motor.

Optionally, the housing is L-shaped.

Optionally, the ear-washing assembly further includes a battery. The battery is arranged within the housing and electrically connected to the control plate.

Optionally, the ear-washing assembly further includes a gravity ball. The gravity ball is arranged at one end of the water inlet pipe extending into the water storage tank.

The present invention also proposes a visual aurilave, including:
a water storage tank provided with a mounting port;
a housing detachably arranged on the mounting port of the water storage tank;
an ear-washing assembly including a control plate arranged in the housing, a water-pumping motor arranged in the housing and electrically connected to the control plate, a water inlet pipe connected to a water inlet of the water-pumping motor at one end thereof, a water outlet pipe connected to a water outlet of the water-pumping motor at one end thereof, a nozzle arranged on the other end of the water outlet pipe, and a transparent water baffle arranged on the water outlet pipe, the other end of the water inlet pipe extending into the water storage tank; and
a camera assembly including a camera arranged on the ear-washing assembly and a signal-transmitting module arranged on the control plate. The camera is electrically connected to the control plate and arranged close to the nozzle. The signal-transmitting module is configured to transmit video signals captured by the camera to a display apparatus.

Optionally, the ear-washing assembly includes a sprinkler seat. One end of the sprinkler seat is fixed in the housing and communicated with the water outlet pipe, and the other end of the sprinkler seat projects out of the housing. The nozzle is arranged at one end of the sprinkler seat located outside the housing.

Optionally, the sprinkler seat is opened and provided with a mounting channel. The camera is arranged in the mounting channel.

Optionally, the camera module includes the camera and a plurality of LED lamp beads provided on the camera and arranged in a circular shape.

Optionally, the ear-washing assembly further includes a front cover arranged on the housing and an LED lamp plate arranged within the front cover and emitting light in a direction toward the nozzle. A middle of the front cover is opened and provided with a fixing hole for fixing the sprinkler seat. The front cover is opened and provided with a light-transmitting hole for accommodating the lamp beads of the LED lamp plate.

The front cover is formed with a sealing groove relative to the other side of the LED lamp plate. The light-transmitting hole is located at a bottom of the sealing groove. The aurilave further includes a transparent sealing ring arranged within the sealing groove.

Optionally, the transparent water baffle is in a form of a disk raised in a middle thereof.

Optionally, the ear-washing assembly further includes a rear cover and a power switch arranged on the rear cover. The power switch is electrically connected to the control plate.

Optionally, the ear-washing assembly further includes a key switch arranged on the housing for controlling the water-pumping motor.

Optionally, the housing is L-shaped.

Optionally, the ear-washing assembly further includes a gravity ball. The gravity ball is arranged at one end of the water inlet pipe extending into the water storage tank.

The aurilave according to the technical solution of the present invention includes the water storage tank, the housing, and the ear-washing assembly. The water storage tank is configured to store a cleaning liquid. The housing is detachably arranged on the mounting port of the water storage tank. The ear-washing assembly is arranged in the housing. The control plate is configured to control the water-pumping motor. The water-pumping motor has the water inlet port and the water outlet port. One end of the water inlet pipe is connected to the water inlet port of the water-pumping motor, and the other end of the water inlet pipe extends into the water storage tank. One end of the water outlet pipe is connected to the water outlet port of the water-pumping motor, and the other end of the water outlet pipe extends out of the housing. The nozzle is arranged at one end of the water outlet pipe extending out of the housing. The transparent water baffle is sleeved on the water outlet pipe near the nozzle. The water-pumping motor pumps the cleaning liquid in the water storage tank and sprays the cleaning liquid via the water outlet pipe to clean an inner side of an ear, which effectively prevents damage to the ear canal and improves safety of ear-canal cleaning.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

In order to explain embodiments of the present invention or the technical solutions in the prior art more clearly, the following briefly introduces the drawings that need to be used in the embodiments or the prior art. Obviously, the drawings in the following description are only some of embodiments of the present invention. Those skilled in the art may obtain other drawings based on structures shown in these drawings without creative labor.

FIG. 1 is a cross-sectional schematic diagram of an aurilave according to a first preferred embodiment of the present invention.

FIG. 2 is a schematic structural diagram of an aurilave according to the above first preferred embodiment of the present invention.

FIG. 3 is a cross-sectional schematic diagram of a visual aurilave according to a second preferred embodiment of the present invention.

FIG. 4 is a schematic structural diagram of the visual aurilave according to the above second preferred embodiment of the present invention.

FIG. 5 is a schematic structural exploded diagram of the visual aurilave according to the above second preferred embodiment of the present invention.

FIG. 6 is a schematic structural diagram of a camera assembly of the visual aurilave according to the above second preferred embodiment of the present invention.

FIG. 7 is a perspective view of an aurilave according to a third preferred embodiment of the present invention.

FIG. 8 is a cross-sectional view of the aurilave according to the above third preferred embodiment of the present invention.

FIG. 9A is another cross-sectional view of the aurilave according to the above third preferred embodiment of the present invention.

FIG. 9B is another cross-sectional view of the aurilave according to the above third preferred embodiment of the present invention.

FIG. 10 is another cross-sectional view of the aurilave according to the above third preferred embodiment of the present invention.

FIG. 11 is another perspective view of the aurilave according to the above third preferred embodiment of the present invention.

FIG. 12 is an explosive view of the aurilave according to the above third preferred embodiment of the present invention.

FIG. 13 is another explosive view of the aurilave according to the above third preferred embodiment of the present invention.

FIG. 14 is a perspective view of an aurilave according to a fourth preferred embodiment of the present invention.

FIG. 15 is another perspective view of the aurilave according to the above fourth preferred embodiment of the present invention.

FIG. 16 is cross-sectional view of the aurilave according to the above fourth preferred embodiment of the present invention.

FIG. 17 is another cross-sectional view of the aurilave according to the above fourth preferred embodiment of the present invention.

FIG. 18 is another perspective view of the aurilave according to the above fourth preferred embodiment of the present invention.

FIG. 19 is another perspective view of the aurilave according to the above fourth preferred embodiment of the present invention.

FIG. 20 is an explosive view of the aurilave according to the above fourth preferred embodiment of the present invention.

FIG. 21 is another explosive view of the aurilave according to the above fourth preferred embodiment of the present invention.

FIG. 22 is a partial cross-sectional view of the aurilave according to the above fourth preferred embodiment of the present invention.

FIG. 23 is another cross-sectional view of the aurilave according to the above fourth preferred embodiment of the present invention.

The realization, functional features, and advantages of the present invention will be described further with reference to the drawings.

### DETAILED DESCRIPTION OF THE PREFERRED EMBODIMENT

The following clearly and completely describes the technical solutions in embodiments of the present invention in conjunction with the drawings in the embodiments of the present invention. Obviously, the described embodiments are only a part of the embodiments of the present invention, rather than all embodiments. Based on the embodiments of the present invention, all other embodiments obtained by those skilled in the art without creative labor shall fall within the protection scope of the present invention.

It should be noted that if the embodiments of the present invention involve directional indications (such as up, down, left, right, front, back . . . ), the directional indications are only used to explain a relative position relationship and movement among various components under a certain posture (as shown in the drawings). If a specific posture changes, the directional indication also changes accordingly.

In addition, if there are descriptions of terms such as “first”, “second” and the like in the embodiments of the present invention, the descriptions of the terms such as “first”, “second” and the like are merely intended for a purpose of description, and shall not be understood as an indication or implication of relative importance or implicit indication of a quantity of indicated technical features. Therefore, the features defined with “first” and “second” may explicitly or implicitly include at least one of the features. In addition, the meaning of “and/or” in the whole text is to include three parallel schemes. Taking “A and/or B” as an example, “A and/or B” includes scheme A, or scheme B, or a scheme that A and B are satisfied at the same time. In addition, the technical solutions of various embodiments can be combined with each other, but should be based on what can be achieved by those skilled in the art. When a combination of technical solutions is contradictory or cannot be achieved, it should be considered that such a combination of technical solutions does not exist, and also does not fall within the scope of protection required by the present invention.

It can be understood that the term “one” should be understood as “at least one” or “one or more”, that is, in one embodiment, the number of elements can be one, while in another embodiment, the number of elements can be multiple. The term “one” cannot be understood as a limitation on the quantity.

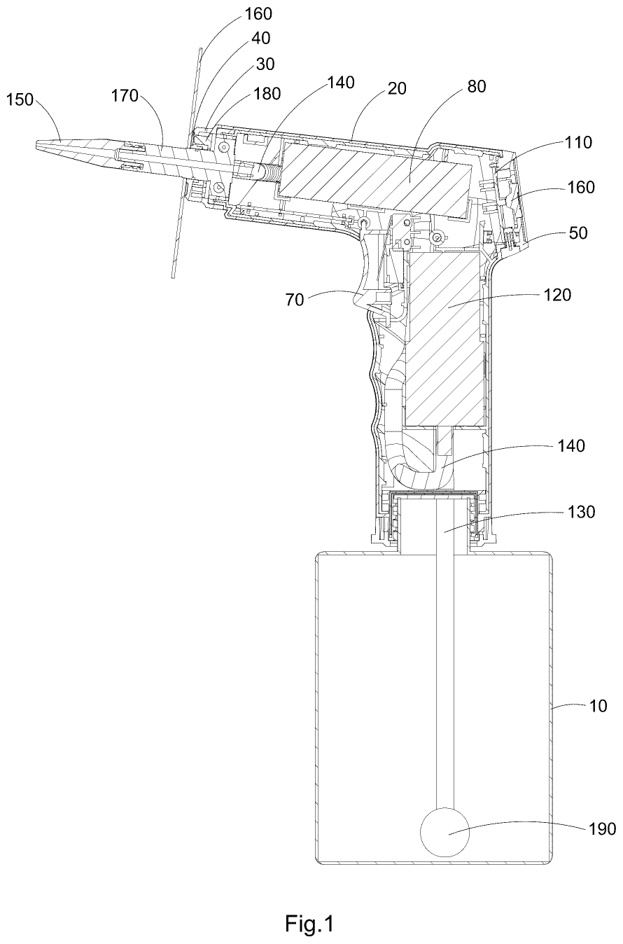

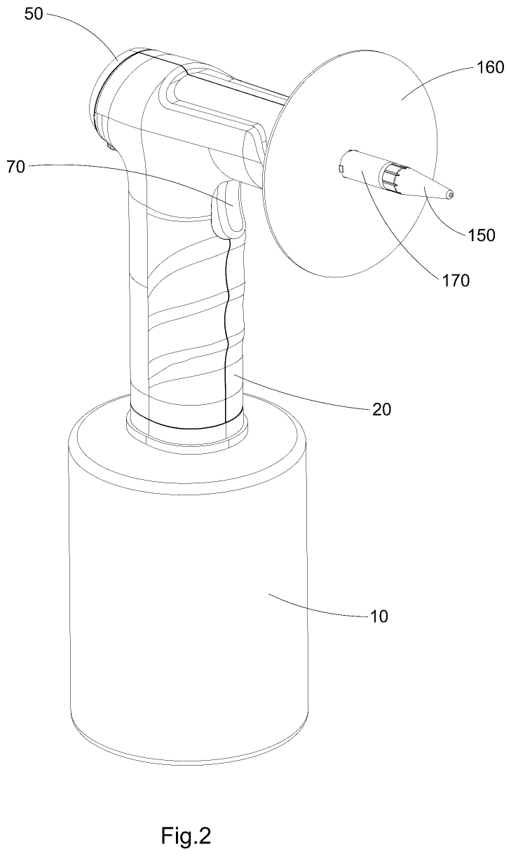

According to a first preferred embodiment of the present invention, as shown in FIGS. 1 to 2, the aurilave includes:
a water storage tank 10 provided with a mounting port;
a housing 20 detachably arranged on the mounting port of the water storage tank 10; and
an ear-washing assembly including a control plate 110 arranged in the housing 20, a water-pumping motor 120 arranged in the housing 20 and electrically connected to the control plate 110, a water inlet pipe 130 connected to a water inlet port of the water-pumping motor 120 at one end thereof, a water outlet pipe 140 connected to a water outlet port of the water-pumping motor 120 at one end thereof, a nozzle 150 arranged on the other end of the water outlet pipe 140, and a transparent water baffle 160 arranged on the water outlet pipe 140. The other end of the water inlet pipe extends into the water storage tank.

According to this first preferred embodiment, the water storage tank 10 is configured to store a cleaning liquid. A body of the water storage tank 10 is opened and provided with a mounting port for mounting the housing 20 and for storing and discharging the cleaning liquid.

The housing 20 is the housing 20 of a hand-held portion of the aurilave, and configured to provide a mounting position for the ear-washing assembly. The housing 20 can be detachably arranged on the mounting port of the water storage tank 10. It can be understood that the housing 20 can be threadedly connected to the water storage tank 10, and can also be snap-fastened to the water storage tank 10. Specifically, no limitation is provided herein. Additionally, the housing 20 may be mounted on a top of the water storage tank 10, at a side wall of the water storage tank 10 or at a bottom of the water storage tank 10. Specifically, no limitation is provided herein. Preferably, the housing 20 is mounted on the top of the water storage tank 10 and threadedly connected to the water storage tank 10. Specifically, a bottom end of the housing 10 is provided with internal threads. The top of the water storage tank 10 protrudes and is provided with a threaded boss. The housing 20 is threadedly connected to the threaded boss so that the housing 20 and the water storage tank 10 are relatively fixed. It needs to be explained that an opening of the water storage tank 10 is located on a top of the threaded boss so that the housing 20 and the water storage tank 10 are communicated with each other.

The ear-washing assembly is a power transmission device of the aurilave, and is configured to apply a driving force to the cleaning liquid in the water storage tank 10 and discharge the cleaning liquid, thereby achieving an effect of flushing an ear canal. Specifically, the control plate 110 is arranged in the housing 20 and configured to be connected to a power source and control the water-pumping motor 120. The water-pumping motor 120 has the water inlet port and the water outlet port. One end of the water inlet pipe 130 is connected to the water inlet port of the water-pumping motor 120, and the other end of the water inlet pipe extends from the mounting port of the water storage tank into the water storage tank 10 so that the water-pumping motor 120 pumps the cleaning liquid in the water storage tank 10. One end of the water outlet pipe 140 is connected to the water outlet port of the water-pumping motor 120, and the other end of the water outlet pipe extends out of the housing 20 and is configured to spray the cleaning liquid. The water outlet end of the water outlet pipe 140 is provided with a nozzle 150. The nozzle 150 is configured to reduce an aperture of the water outlet pipe 140 to improve a water pressure strength. In addition, the transparent water baffle 160 is sleeved on the water outlet pipe 140 close to the nozzle 150. The transparent water baffle 160 is configured to fend against water drops splashed from the ear so that the user can conveniently observe a cleaning condition within the ear canal.

The aurilave according to the technical solution of the present invention includes the water storage tank 10, the housing 20, and the ear-washing assembly. The water storage tank 10 is configured to store the cleaning liquid. The housing 20 is detachably arranged on the mounting port of the water storage tank 10. The ear-washing assembly is arranged in the housing 20. The control plate 110 is configured to control the water-pumping motor 120. The water-pumping motor 120 has the water inlet port and the water outlet port. One end of the water inlet pipe 130 is connected to the water inlet port of the water-pumping motor 120, and the other end of the water inlet pipe 130 extends into the water storage tank 10. One end of the water outlet pipe 140 is connected to the water outlet port of the water-pumping motor 120, and the other end of the water outlet pipe extends out of the housing 20. The nozzle 150 is arranged at one end of the water outlet pipe 140 extending out of the housing 20. The transparent water baffle 160 is sleeved on the water outlet pipe 140 near the nozzle 150. The water-pumping motor 120 pumps the cleaning liquid in the water storage tank 10 and sprays the cleaning liquid via the water outlet pipe 140 to clean an inner side of an ear, which effectively prevents damage to the ear canal and improves safety of ear-canal cleaning.

Furthermore, as shown in FIGS. 1 to 2, the ear-washing assembly includes a sprinkler seat 170. One end of the sprinkler seat 170 is fixed in the housing 20 and communicated with the water outlet pipe 140, and the other end of the sprinkler seat 170 protrudes out of the housing 20. The nozzle 150 is arranged at one end of the sprinkler seat 170 located outside the housing 20. According to this first preferred embodiment, the sprinkler seat 170 is made of a rigid material. An inner part of the sprinkler seat is formed with a water guiding channel 172. One end of the sprinkler seat 170 is fixed in the housing 20. One end of the water outlet pipe 140 is fixed on the sprinkler seat 170 and communicated with the water guiding channel 172 of the sprinkler seat 170. The other end of the sprinkler seat 170 extends out of the housing 20 and is configured to spray water. The nozzle 150 is arranged at one end of the sprinkler seat 170 extending out of the housing 20. Arrangement of the sprinkler seat 170 makes a water outlet end of the aurilave form a stable water flow direction, thus effectively improving water flow stability of the aurilave, and improving practicality of the aurilave.

Furthermore, as shown in FIGS. 1 to 2, the ear-washing assembly further includes a front cover 30 arranged on the housing 20 and an LED lamp plate 180 arranged within the front cover 30 and emitting light in a direction toward the nozzle 150. A middle of the front cover 30 is opened and provided with a fixing hole 32 for fixing the sprinkler seat 170. The front cover 30 is opened and provided with a light-transmitting hole 33 for accommodating lamp beads of the LED lamp plate 180. According to this first preferred embodiment, the housing 20 includes a left housing and a right housing. The left housing and the right housing are snap-fitted to form a mounting space of the ear-washing assembly. The front cover 30 is arranged at a water outlet end of the housing 20 to improve structural stability of the left housing and the right housing. The LED lamp plate 180 is configured to improve brightness in the ear canal and facilitate the user to understand a cleaning situation in the ear canal. Specifically, a middle of the front cover 30 is opened and provided with a fixing hole 32 for fixing the sprinkler seat 170 to improve mounting stability of the sprinkler seat 170. Additionally, the front cover 30 is also opened and provided with the light-transmitting hole 33 for accommodating the lamp beads of the LED lamp plate 180 around an axial direction of the fixing hole 32. The lamp beads are located in the light-transmitting hole 33 and emit light in a direction of the nozzle 150 to illuminate the ear canal, so that the user can more clearly observe a situation in the ear canal. Therefore, the user conveniently cleans the ear canal, thereby effectively improving the practicality of the aurilave.

In addition, a waterproof adhesive ring is also arranged in the fixing hole 32. The waterproof adhesive ring is sleeved on the sprinkler seat 170 and abuts against an inner wall of the fixing hole 32, thereby effectively improving sealing of the aurilave.

Furthermore, the front cover 30 is formed with a sealing groove 31 relative to the other side of the LED lamp plate 180 corresponding to the lamp beads of the LED lamp plate 180. The light-transmitting hole is located at a bottom of the sealing groove 31. A transparent sealing ring 40 is arranged in the sealing groove 31, thereby effectively improving the sealing of the aurilave without affecting a lighting effect of the LED lamp plate 180.

Furthermore, the transparent water baffle 160 is in a form of a disk raised in a middle thereof. According to this first preferred embodiment, a middle of the transparent water baffle 160 is raised to achieve a magnifying effect similar to that of a convex lens, thereby facilitating the user to know the cleaning situation within the ear canal. The transparent water baffle 160 is arranged in a disk shape, so that when water drops are splashed on the transparent water baffle 160, the water drops are converged towards a lower middle part of the transparent water baffle 160, thereby reducing coagulation of the water drops on the transparent water baffle 160 to affect the user's viewing. This effectively improves the practicality of the aurilave.

Furthermore, the ear-washing assembly further includes a rear cover 50 and a power switch 60 arranged on the rear cover 50. The power switch 60 is electrically connected to the control plate 110. According to this first preferred embodiment, the rear cover 50 is configured to improve structural stability of the housing 20 and provide a mounting position for the power switch 60. The power switch 60 is arranged on the rear cover 50 and electrically connected to the control plate 110 to control a power on/off of the water-pumping motor 120.

Furthermore, the ear-washing assembly further includes a key switch 70 arranged on the housing 20 for controlling the water-pumping motor 120. According to this first preferred embodiment, the housing 20 is opened and provided with a mounting hole. The key switch 70 is arranged in the mounting hole for controlling startup or shutdown of the water-pumping motor 120, thereby controlling spraying or shutdown of water of the aurilave. This making it convenient for the user to use, and effectively improves the practicality of the aurilave.

Furthermore, the housing 20 is L-shaped. According to this first preferred embodiment, the housing 20 has two functions. One function is that the user needs to hold the housing 20, and the other function is that the housing 20 needs to be mounted with the ear-washing assembly for water spraying and cleaning. According to this first preferred embodiment, the housing 20 is arranged as an L-shape so that the user can easily hold the housing 20, and that a height of a space of the aurilave is effectively reduced to make it more user-friendly.

Furthermore, the ear-washing assembly further includes a battery 80. The battery 80 is arranged within the housing 20 and electrically connected to the control plate 110. According to this first preferred embodiment, the battery 80 is configured to supply power to the water-pumping motor 120 and the LED lamp plate 180. Specifically, the battery 80 is arranged in the housing 20, is electrically connected to the control plate 110, and stably supplies power to the water-pumping motor 120 and the LED lamp plate 180 via the control plate 110.

Furthermore, the ear-washing assembly further includes a gravity ball 190 arranged at one end of the water inlet pipe 130 extending into the water storage tank 10. According to this first preferred embodiment, since the water inlet pipe 130 is generally a hose, when immersed in a liquid, the water inlet pipe floats on a surface of the liquid due to a buoyant force of the liquid, thereby affecting pumping efficiency of the water-pumping motor 120. Therefore, According to this first preferred embodiment, one end of the water inlet pipe 130 extending into the water storage tank 10 is provided with the gravity ball 190, thereby effectively maintaining a water inlet end of the water inlet pipe 130 at a bottom of the water storage tank 10. This further maintains the pumping efficiency of the water-pumping motor 120, and effectively improves the practicality of the aurilave.

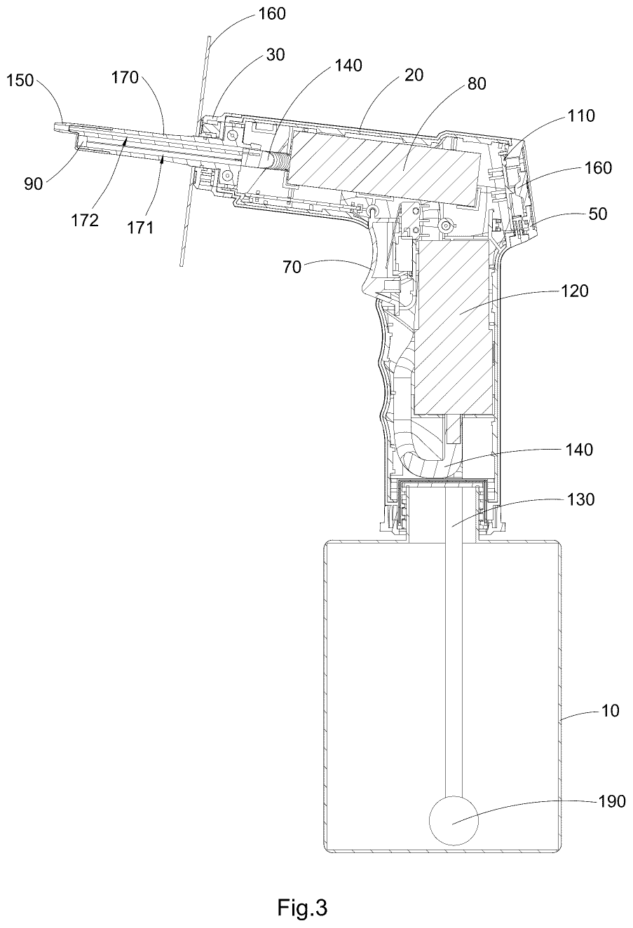

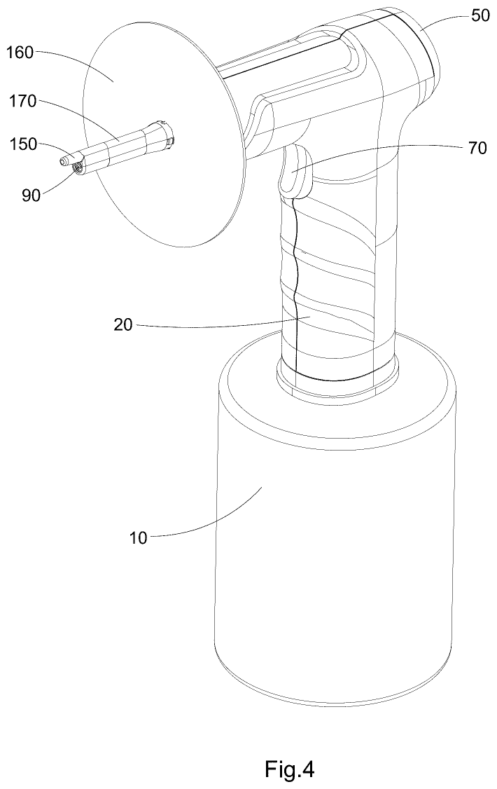

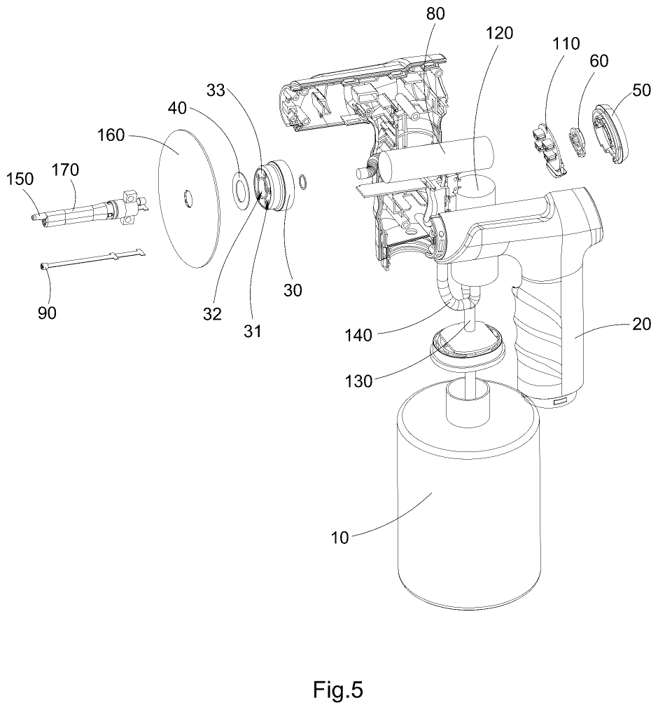

FIGS. 3 to 5 illustrates a visual aurilave according to a second preferred embodiment of the present invention. The visual aurilave includes: a water storage tank 10 provided with a mounting port;
a housing 20 detachably arranged on the mounting port of the water storage tank 10;
an ear-washing assembly including a control plate 110 arranged in the housing 20, a water-pumping motor 120 arranged in the housing 20 and electrically connected to the control plate 110, a water inlet pipe 130 connected to a water inlet port of the water-pumping motor 120 at one end thereof, a water outlet pipe 140 connected to a water outlet port of the water-pumping motor 120 at one end thereof, a nozzle 150 arranged on the other end of the water outlet pipe 140, and a transparent water baffle 160 arranged on the water outlet pipe 140. The other end of the water inlet pipe 130 extends into the water storage tank 10, and
a camera assembly 90 including a camera 91 arranged on the ear-washing assembly and a signal-transmitting module arranged on the control plate 110. The camera 91 is electrically connected to the control plate 110 and arranged close to the nozzle 150. The signal-transmitting module is configured to transmit video signals captured by the camera 91 to a display apparatus.

It is to be explained that specific structures of the water storage tank 10, the housing 20, and the ear-washing assembly of the visual aurilave are the same as features of the corresponding embodiments of the aurilave described above, and thus are not repeated.

The camera assembly 90 is used for the user to observe the situation in the ear canal. Specifically, the camera 91 is arranged on the ear-washing assembly and close to a nozzle 150. Additionally, a signal-transmitting module is arranged on a control plate 110110. The camera 91 shoots a video image in the ear canal to form video signals and transmits the video signals to the control plate 110. The control plate 110 transmits the video signals to a display apparatus for display via the signal-transmitting module, so that the user can view cleaning conditions of various corners in the ear canal in real time on the display apparatus, thereby making it more convenient for the user to clean the ear canal.

A transmission mode of the signal-transmitting module can be wired transmission or wireless transmission, which are not limited specifically here.

Furthermore, the sprinkler seat 170 is opened and provided with a mounting channel 171. The camera 91 is arranged within the mounting channel 171 to prevent the camera from being eroded by a cleaning liquid. The mounting channel 17 is specifically arranged below the water guiding channel 172. It needs to be explained that the mounting channel 171 is arranged parallel to the water guiding channel 172. The mounting channel 171 has a length less than that of the water guiding channel 172. The camera is arranged at one end of the mounting channel 171 close to the nozzle 150150, so that the user can observe a cleaning condition of the ear canal flushed by the nozzle 150150. Therefore, the user can adjust a cleaning position according to the cleaning condition at any time, thereby effectively improving cleaning efficiency of the aurilave.

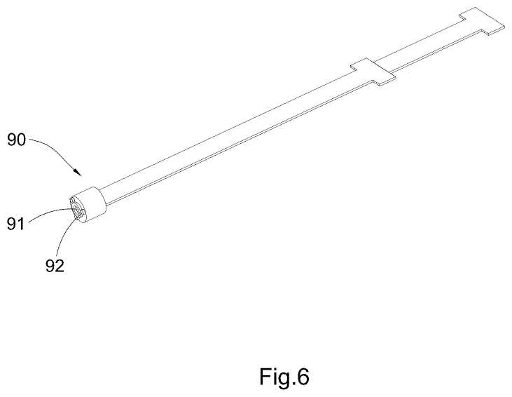

Furthermore, as shown in FIG. 6, the camera assembly 90 further includes a plurality of LED lamp beads 92 arranged on the camera 91. The LED lamp beads 92 are provided on the camera 91 and arranged in a circular shape.

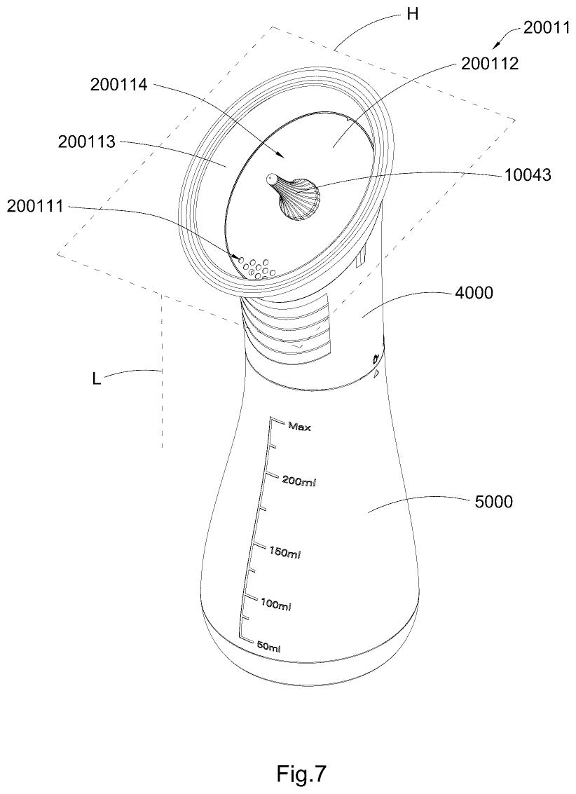

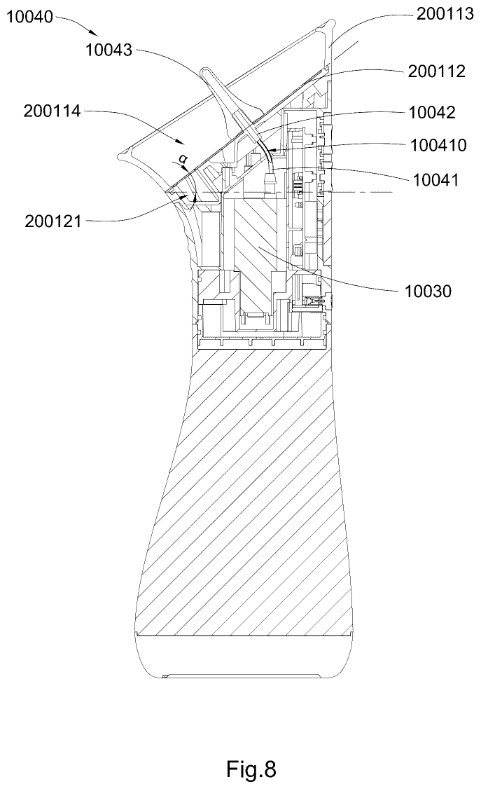

FIGS. 7 to 8 illustrate an aurilave according to a third preferred embodiment of the present invention. The aurilave comprises a liquid supplying assembly 1000 and a waste liquid receiving assembly 2000, wherein the liquid supplying assembly 1000 is configured to provide cleaning fluid to the ear canal. After the cleaning fluid is injected into the ear canal, it flushes out cerumen and debris, which are then collected by the waste liquid receiving assembly 2000. This closed-loop design ensures hygiene and prevents spillage.

The liquid supplying assembly 1000 comprises a first liquid storage chamber 10010, a liquid inlet pipe 10020, a liquid pump 10030 and an ear rinsing assembly 10040. The first liquid storage chamber 10010 defines a first cavity 10011 for storing cleaning fluid, which is delivered through the liquid inlet pipe 10020 to the liquid pump 10030. The liquid pump 10030 pressurizes the cleaning fluid and directs it to the ear rinsing assembly 10040. The ear rinsing assembly 10040 guides the cleaning fluid into the ear canal and clean the ear canal by gently irrigating the accumulated cerumen, ensuring effective removal without damaging the delicate skin or eardrum.

The ear rinsing assembly 10040 comprises a liquid outlet pipe 10041, an extending element 10042, and a jet 10043. The liquid outlet pipe 10041 conveys the pressurized cleaning fluid from the liquid pump 10030 to the extending element 10042, which is designed to safely position the jet 10043 near the entrance of the ear canal. The jet 10043 emits a controlled stream of cleaning fluid that gently flushes out cerumen and debris, with adjustable pressure to suit individual sensitivity.

The waste fluid receiving assembly 2000 collects the spent cleaning fluid and dislodged cerumen after irrigation, channeling it away from the ear and into a sealed containment unit to prevent leakage or contamination. The waste liquid receiving assembly 2000 comprises a liquid collection assembly 20010, a liquid reflow pipe 20020, and a second liquid storage chamber 20030 defining a second cavity 20031. The liquid collection assembly 20010 collects the effluent from the ear region, forming a safe capture zone that prevents splashing or spillage during operation. The effluent collected by the liquid collection assembly 20010 is guided through the liquid reflow pipe 20020 into the second cavity 20031 of the second liquid storage chamber 20030.

The first cavity 10011 and the second cavity 20031 are fluidically isolated from each other to maintain a closed-loop system, ensuring that the cleaning fluid remains segregated from the waste stream. This separation enhances hygiene, prevents cross-contamination.

The aurilave further comprises a control system 30000 electrically connected to the liquid pump 10030, so as to control the operation of the liquid pump 10030 in a programmable manner, enabling precise regulation of cleaning fluid flow rate and duration.

The control system 30000 comprises a power supply module 3001, a main control unit 3002, and a display module 3003, wherein the main control unit 3002 is electrically connected to the power supply module 3001 and the display module 3003 to coordinate system operations. The power supply module 3001 comprises a battery 80 and a charging interface 81 electrically connected to the battery 80 for charging connection.

The display module 3003 display the signal that indicates the power level of the battery 80. The control system 3002 is electrically connected to the liquid pump 10030, so as to control the start, the stop, and the flow rate of the liquid pump 10030.

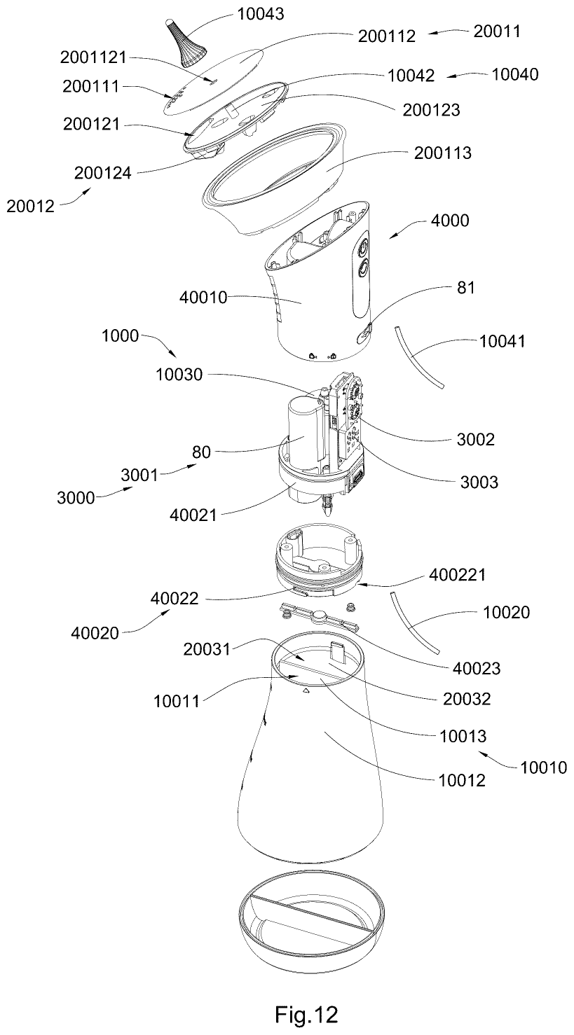

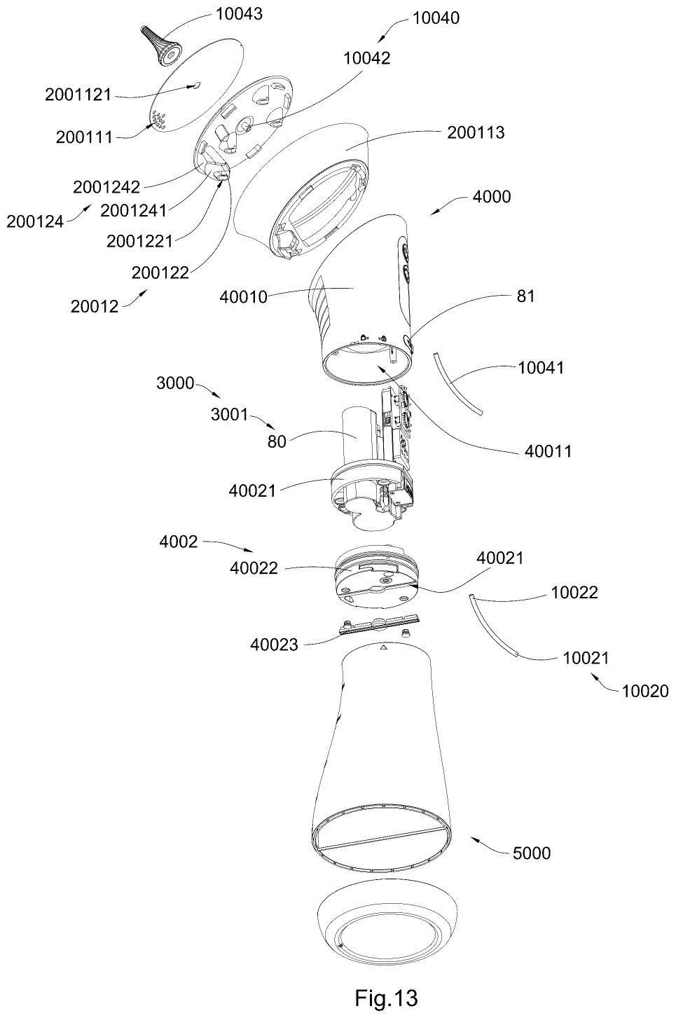

As can be shown in FIGS. 12 and 13, the aurilave further comprises a shell 4000, wherein the shell 4000 comprises a shell body 40010 and a lower cover 40020, wherein the shell body 40010 defines a shell space 40011. The lower cover 40020 is detachably installed to the shell body 40010, thereby enclosing the shell space 40011.

According to this third preferred embodiment of the present invention, the first liquid storage chamber 10010 and the second liquid storage chamber 20030 are integrally formed together. In detail, the first liquid storage chamber 10010 comprises a first chamber body 10012 and a separating wall 10013, wherein the first chamber body 10012, together with the separating wall 10013, defines the first cavity 10011. The liquid storage chamber 20030 comprises a second chamber body 20032, wherein the second chamber body 20032, together with the separating wall 10013, defines the second cavity 20031. In more detail, the first chamber body 10012, together with the second chamber body 20032 forms a jar 5000 defining a jar cavity 5001, wherein the jar 5000 can be detachably installed to the lower cover 40020 of the shell 4000. The separating wall 10013 is integrally formed within the jar cavity 5001, dividing the jar cavity 5001 into the first cavity 10011 and the second cavity 20031, ensuring structural integrity and leak prevention between the two chambers.

The jar 5000 has an installation wall 5002 that extends upwardly, wherein the installation wall 5002 is configured to engage with the lower cover 40020. The installation wall 5002 is higher than the separating wall 10013, ensuring a secure seal when the jar 5000 is mounted to the shell 4000. When the lower cover 40020 of the shell 4000 is secured onto the installation wall 5002, a peripheral seal is formed between the lower cover 4002 and the installation wall 5002, and the lower cover 40020 presses against the top edge of the separating wall 10013, compressing it slightly to simultaneously cover the first cavity 10011 and the second cavity 20031, thereby preventing the waste liquid in the second cavity 20031 from mixing with the clean liquid in the first cavity 10011.

The lower cover 40020 defines a waste liquid hole 40021 and a clean liquid hole 40022, wherein the waste liquid hole 40021 align with the second cavity 20031 and allows waste liquid to pass through while the clean liquid hole 40022 aligns with the first cavity 10011, enabling clean liquid flow.

According to this third preferred embodiment of the present invention, the lower cover 40020 comprises a first cover body 40021 used to cover the shell body 40010, and a second cover body 40022 connected to the first cover body 40021, wherein the second cover body 40022 is configured to sealably engage with the jar 5000. The second cover body 40022 defines a sealing groove 400221. The lower cover 40020 further comprises a sealing strip 40023 disposed within the sealing groove 400221, wherein the sealing strip 40023 is elastically compressible to ensure a tight seal between the second cover body 40022 and the separating wall 10013.

The liquid inlet pipe 10020 is extended into the first cavity 10011. When the clean liquid is stored within the first cavity 10011, a first inlet pipe end 10021 of the liquid inlet pipe 10020 is submerged below the liquid level, a second inlet pipe end 10022 of the liquid inlet pipe 10020 is communicated with the liquid pump 10030, which draws the clean liquid through the submerged first inlet pipe end 10021 and delivers the clean liquid to the ear rinsing assembly 10040, and end the clean liquid is expelled through the ear rinsing assembly 10040 into the ear canal.

The clean liquid effectively flushes debris and cerumen from the ear canal, and flows outward carrying the dislodged particles as waste liquid. The waste liquid flows from the ear canal into the liquid collection assembly 20010, and then flows through the liquid reflow pipe 20020 into the second liquid storage chamber 20030.

According to this third preferred embodiment, the liquid collection assembly 20010 comprises a liquid collection tray 20011 and a temporary storage chamber body 20012, wherein the waste liquid flows out of the ear canal is collected by the liquid collection tray 20011, and then guided into the temporary storage chamber body 20012, and then guided through the liquid reflow pipe 20020 into the second liquid storage chamber 20030. In detail, the liquid collection tray 20011 has at least one flow hole 200111 at a lower portion thereof, allowing the collected waste liquid to flow into the temporary storage chamber body 20012. The temporary storage chamber body 20012 defines a temporary storage chamber 200121 for temporarily holding the waste liquid before it is guided through the liquid reflow pipe 20020. A liquid outlet hole body 200122 is provided at a bottom portion of the temporary storage chamber body 20012, through which the waste liquid is discharged into the liquid reflowing pipe 20020. The liquid reflowing pipe 20020 securely communicates the temporary storage chamber body 20012 with the second liquid storage chamber 20030, ensuring an efficient transfer of waste liquid from the temporary storage chamber to the second liquid storage chamber 20030, under the force of gravity.

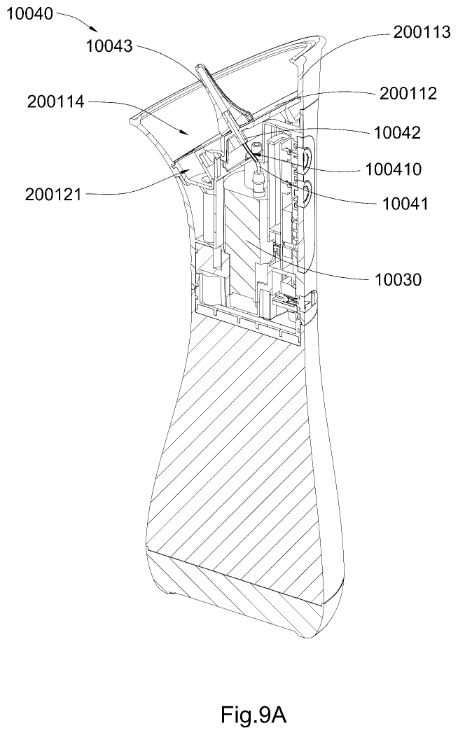

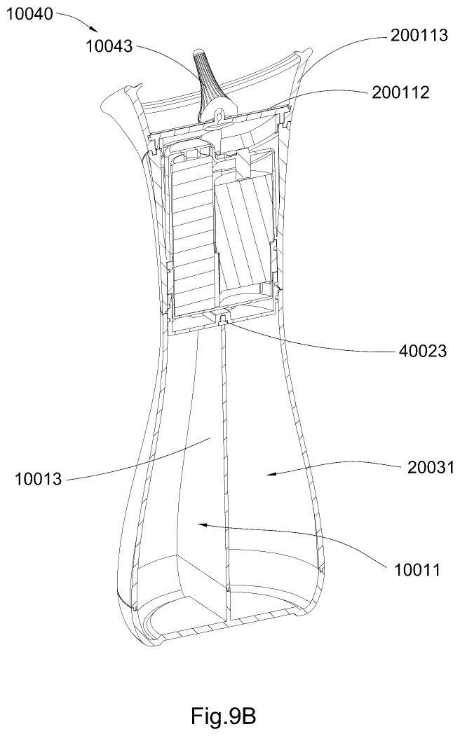

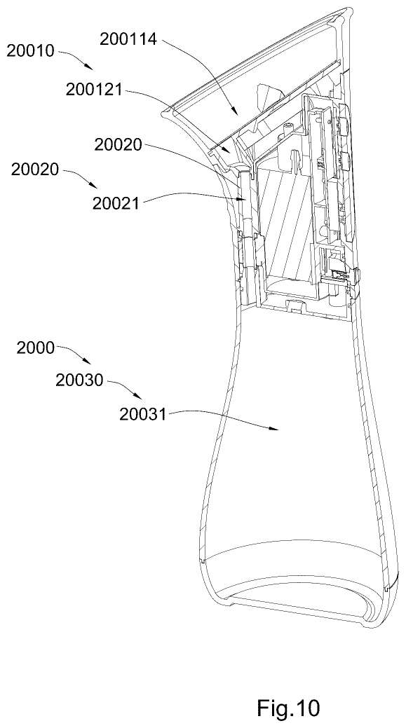

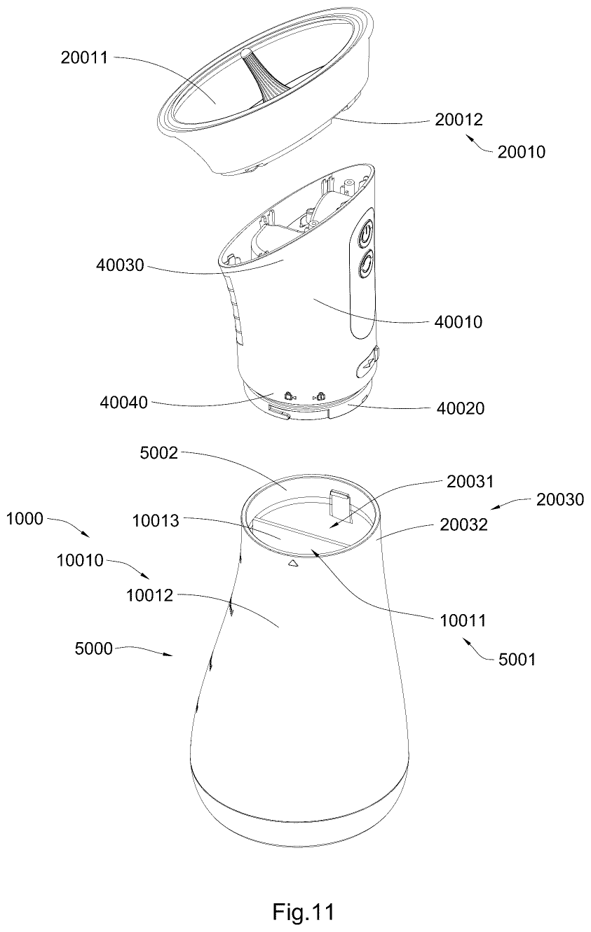

As can be shown in FIGS. 7 to 13, the liquid collection tray 20011 is designed with a incline to facilitate the smooth flow of waste liquid toward the temporary storage chamber body 20012. In detail, the liquid collection tray 20011 comprises a guiding plate 200112 that slopes with respect to a horizontal plane H defined by the aurilave, wherein the horizontal plane H is vertically aligned with an extension direction L defined by the aurilave, wherein with respect to the shell 4000, the jar 5000 is installed at a lower position allow the extension direction L, wherein the liquid collection tray 20011 is installed at an higher position relative to the jar 5000, enabling gravitational drainage from the guiding plate 200112 into the temporary storage chamber body 20012.

According to this third preferred embodiment of the present invention, a tilt angle α between the guiding plate 200112 and the horizontal plane H ranges from 20° to 60°. More preferably, the tilt angle α ranges from 30° to 40°, and most preferably, the tilt angle α is approximately 35°.

The liquid collection tray 20011 further comprises a peripheral flange 200113, wherein the peripheral flange 200113 and the guiding plate 200112 define a collection chamber 200114 that confines the waste liquid and prevents overflow, wherein the peripheral flange 200113 extends upward from the guiding plate 200112 to enclose the lateral boundaries of the collection chamber 200114, ensuring containment prior to gravitational discharge into the temporary storage chamber 200121.

The guiding plate 200112 defines the flow hole 200111 being positioned at a lower elevation while the aurilave being in a usage state, facilitating efficient drainage under gravity.

In detail, in the usage state, the guiding plate 200112 is inclined such that the flow hole 200111 is resided at the lower point, promoting runoff of cleaning liquid into the temporary storage chamber 200121. In more detail, the extending element 10042 of the ear rinsing assembly 10040 is configured to be perpendicular to the guiding plate 200112, and the jet 10043 being installed on the extending element 10042 is perpendicular to the guiding plate 200112. In the usage state, the jet 10043 is inserted into the ear canal at a predetermined angle, ensuring precise delivery of the cleaning liquid. The peripheral flange 200113 surrounds the user's ear and forms a sealed environment to prevent leakage, while the guiding plate 200112 is faced toward the user's ear, forming a protective barrier that prevents splashing, thereby ensuring the waste liquid being efficiently channeled through the flow hole 200111 into the temporary storage chamber 200121.

The extending element 10042 is integrally formed with the temporary storage chamber body 20012 and extends outwardly therefrom. The guiding plate 200112 further has a first through hole 2001121 allows the extending element 10042 to pass through, wherein the surrounding area of the first through hole 2001121 is sealed around the extending element 10042 to prevent fluid leakage.

The guiding plate 200112 and the temporary storage chamber body 20012 are installed together in such a manner that the extending element 10042 passes through the first through hole 2001121.

The guiding plate 200112 covers the temporary storage chamber 20012 in such a manner that the flow hole 200111 communicates directly with the temporary storage chamber 200121, ensuring a seamless transfer of waste liquid without leakage. The liquid outlet hole body 200122 is communicated with the temporary storage chamber 200121 and positioned at the bottom of the temporary storage chamber body 20012, allowing efficient drainage of collected waste liquid.

In detail, the temporary storage chamber body 20012 comprises a plate body 200123 and a plate body 200123 and a temporary storage chamber main body 200124 integrally formed to the plate body 200123, wherein the temporary storage chamber main body 200124 defines the temporary storage chamber 200121. In more detail, the temporary storage chamber main body 200124 comprises a bottom wall 2001241 and a surrounding wall 2001242 extending upwardly from the periphery of the bottom wall 2001241, wherein the bottom wall 2001241 is slanted, so that the liquid outlet hole body 200122 can be positioned at a lower position of the bottom wall 2001241 to ensure complete and rapid discharge of waste liquid under gravity.

The liquid reflow pipe 20020 is integrally formed with shell body 40010 of the shell 4000 and defines a liquid reflow channel 20021 that allows waste liquid to flow back into the second cavity 20031 of the liquid storage chamber 20030.

The liquid outlet hole body 200122 has a liquid outlet hole 2001221 that communicates with the liquid reflow channel 20021, ensuring a seamless transfer of waste liquid from the temporary storage chamber 200121 to the second cavity 20031.

According to other embodiments of the present invention, the aurilave can further comprises a drying assembly, so as to dry the ear canal after cleaning.

According to other embodiments of the present invention, the aurilave can further comprise a heating system electrically connected to the control system 3000, so as to heat the cleaning liquid in the first cavity 10011.

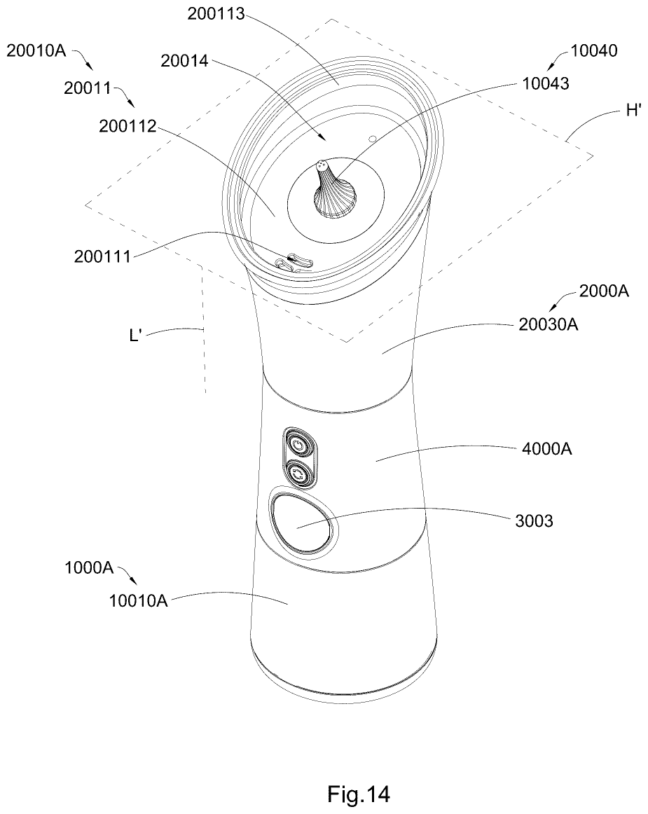

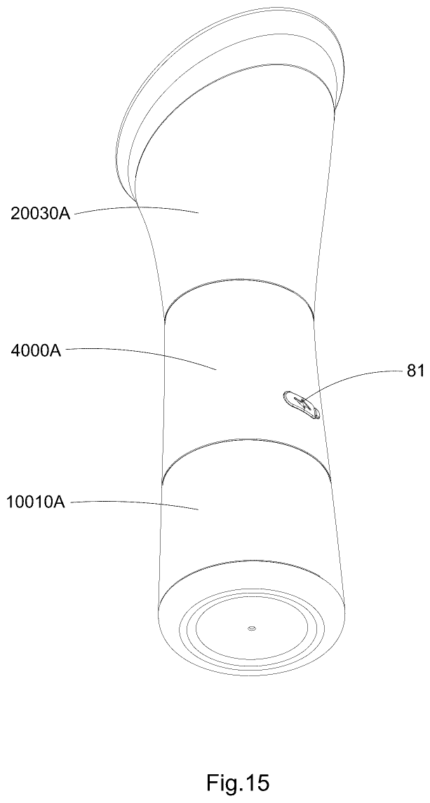

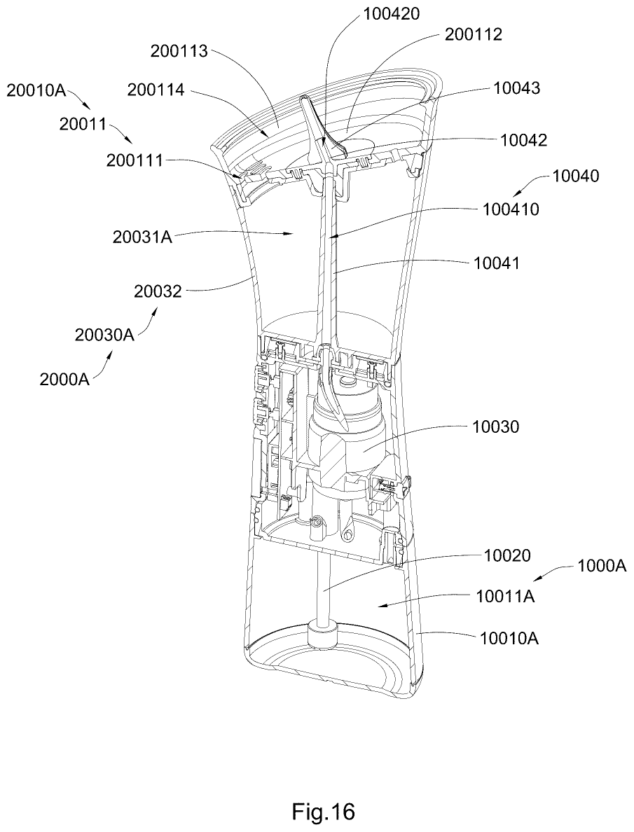

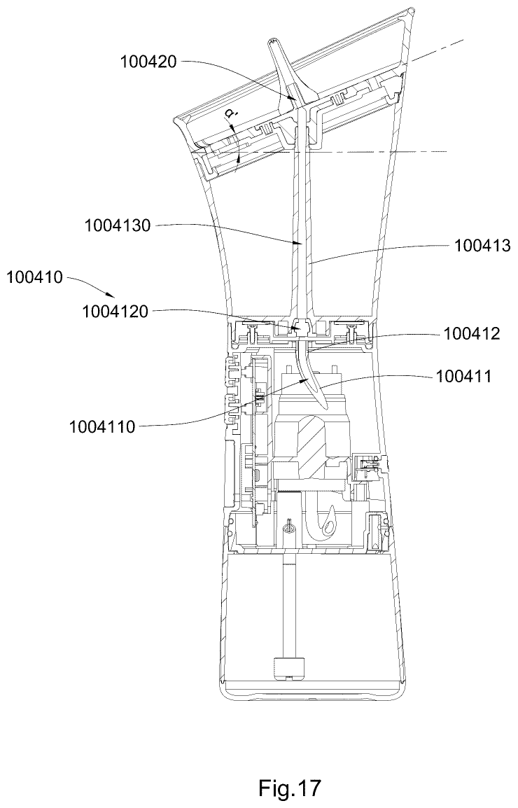

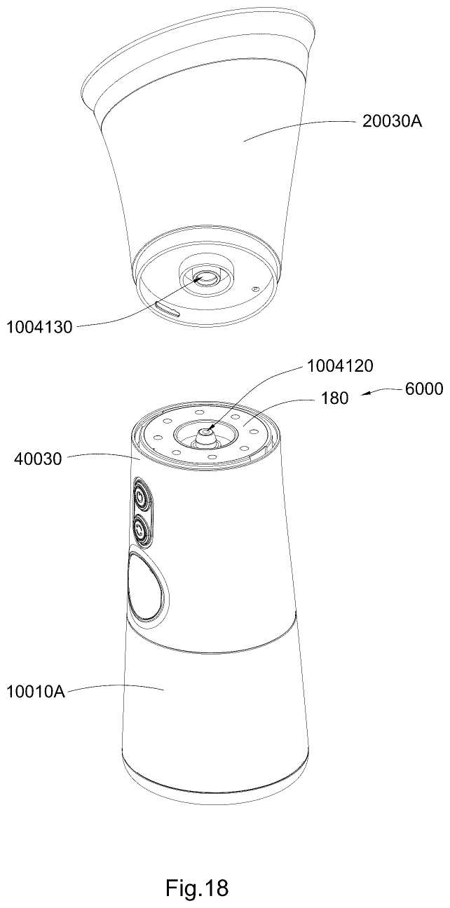

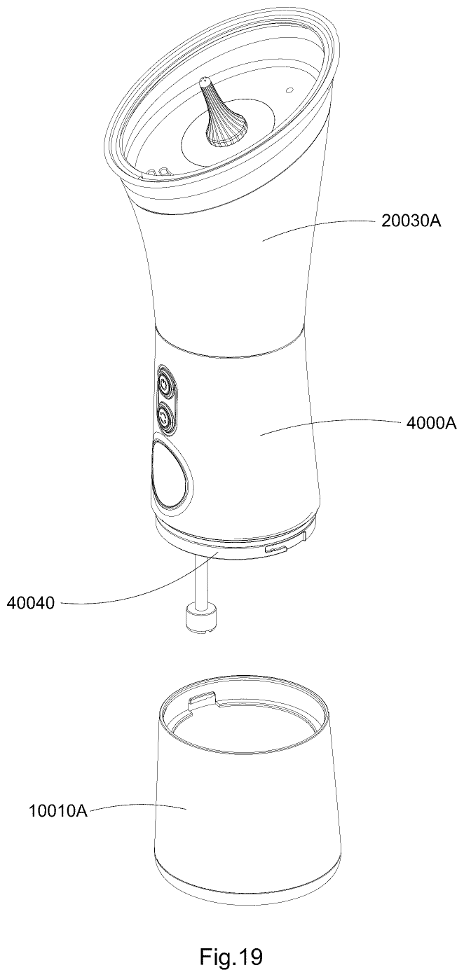

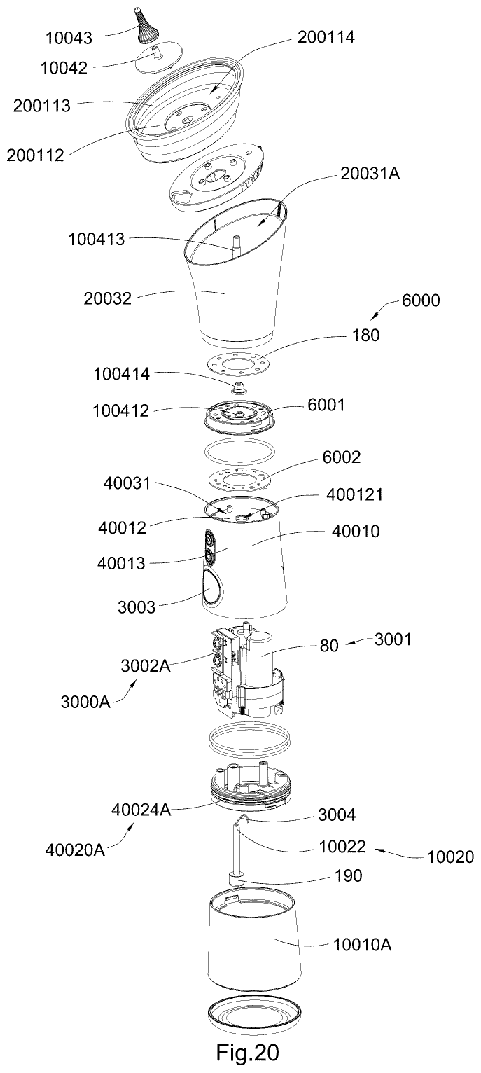

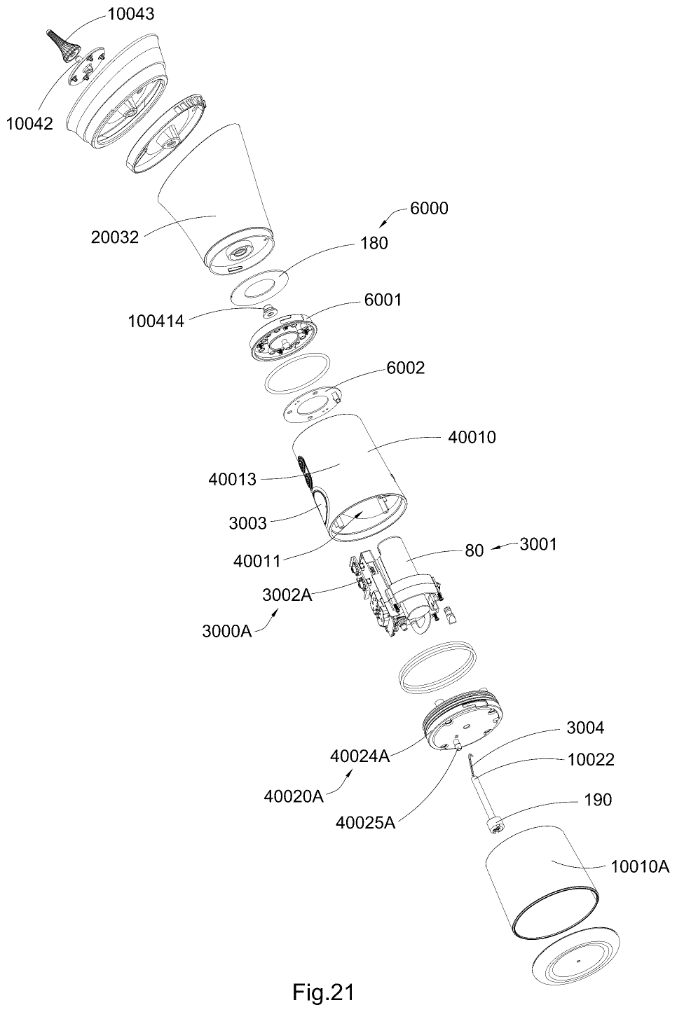

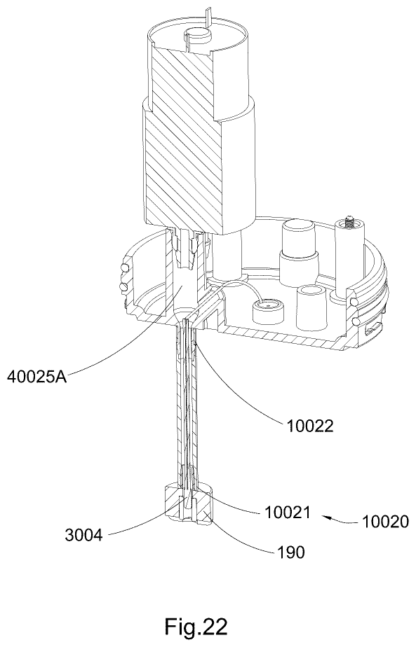

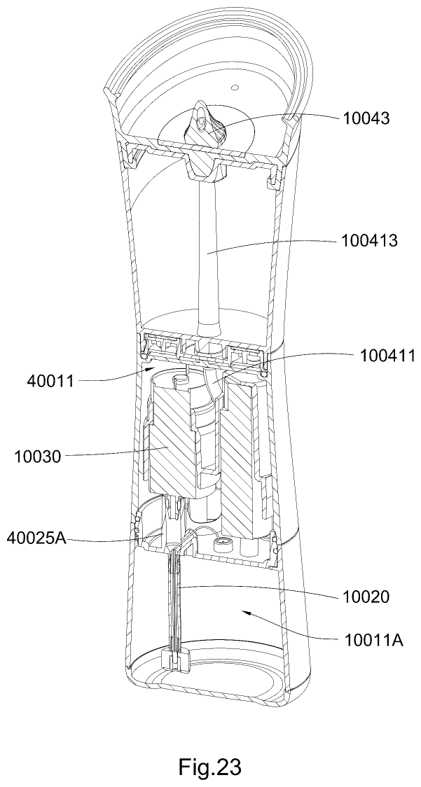

FIGS. 14 to 23 illustrate an aurilave according to a fourth preferred embodiment of the present invention. The aurilave comprises a liquid supplying assembly 1000A and a waste liquid receiving assembly 2000A, wherein the liquid supplying assembly 1000A is configured to provide cleaning fluid to the ear canal. After the cleaning fluid is injected into the ear canal, it flushes out cerumen and debris, which are then collected by the waste liquid receiving assembly 2000A.

The liquid supplying assembly 1000A comprises a first liquid storage chamber 10010A, a liquid inlet pipe 10020, a liquid pump 10030 and an ear rinsing assembly 10040. The first liquid storage chamber 10010A defines a first cavity 10011A for storing cleaning fluid, which is delivered through the liquid inlet pipe 10020 to the liquid pump 10030. The liquid pump 10030 pressurizes the cleaning fluid and directs it to the ear rinsing assembly 10040. The ear rinsing assembly 10040 guides the cleaning fluid into the ear canal and clean the ear canal by gently irrigating the accumulated cerumen, ensuring effective removal without damaging the delicate skin or eardrum.

The ear rinsing assembly 10040 comprises a liquid outlet pipe 10041, an extending element 10042, and a jet 10043. The liquid outlet pipe 10041 conveys the pressurized cleaning fluid from the liquid pump 10030 to the extending element 10042, which is designed to safely position the jet 10043 near the entrance of the ear canal. The jet 10043 emits a controlled stream of cleaning fluid that gently flushes out cerumen and debris, with adjustable pressure to suit individual sensitivity. The jet 10043 is detachably installed to the extending element 10042.

The waste liquid receiving assembly 2000A collects the spent cleaning fluid and dislodged cerumen after irrigation, channeling it away from the ear and into a sealed containment unit to prevent leakage or contamination. The waste liquid receiving assembly 2000A comprises a liquid collection assembly 20010A and a second liquid storage chamber 20030A defining a second cavity 20031A. The liquid collection assembly 20010A collects the effluent from the ear region, forming a safe capture zone that prevents splashing or spillage during operation. The effluent collected by the liquid collection assembly 20010A is guided into the second cavity 20031A of the second liquid storage chamber 20030A.

The first cavity 10011A and the second cavity 20031A are fluidically isolated from each other to maintain a closed-loop system, ensuring that the cleaning fluid remains segregated from the waste stream. This separation enhances hygiene, prevents cross-contamination.

The aurilave further comprises a control system 3000A electrically connected to the liquid pump 10030, so as to control the operation of the liquid pump 10030 in a programmable manner, enabling precise regulation of cleaning fluid flow rate and duration.

The control system 3000A comprises a power supply module 3001, a main control unit 3002A, and a display module 3003, wherein the main control unit 3002A is electrically connected to the power supply module 3001 and the display module 3003 to coordinate system operations. The power supply module 3001 comprises a battery 80 and a charging interface 81 electrically connected to the battery 80 for charging connection.

The display module 3003 display the signal that indicates the power level of the battery 80. The control system 3002 is electrically connected to the liquid pump 10030, so as to control the start, the stop, and the flow rate of the liquid pump 10030.

The aurilave further comprises a shell 4000A, wherein the shell 4000A comprises a shell body 40010 and a lower cover 40020A, wherein the shell body 40010 defines a shell space 40011. The lower cover 40020A is detachably installed to the shell body 40010, thereby enclosing the shell space 40011.

According to this fourth preferred embodiment of the present invention, the first liquid storage chamber 10010A and the second liquid storage chamber 20030A are installed opposite to each other, above and below the shell 4000A. In detail, the first liquid storage chamber 10010A is installed at a top portion 40030 of the shell 4000A, while the second liquid storage chamber 20030A is installed at a bottom portion 40040 of the shell 4000A.

The lower cover 40020A defines a cleaning liquid hole 40022A, wherein the cleaning liquid hole 40022A communicating with the liquid pipe 10020.

In detail, the lower cover 40020A comprises a cover main body 40024A and a cleaning liquid hole body 40025A defining the cleaning liquid hole 40022A, wherein the cleaning liquid hole body 40025A is integrally formed with the cover main body 40024A, so as to prevent the liquid from leaking. The liquid inlet pipe 10020 is extended into the first cavity 10011A. When the cleaning liquid is stored within the first cavity 10011A, a first inlet pipe end 10021 of the liquid inlet pipe 10020 is extended to the cleaning liquid, a second inlet pipe end 10022 of the liquid inlet pipe 10020 is communicated with the cleaning liquid hole body 40025A. The cleaning liquid hole body 40025A is communicated with the liquid pump 10030, which draws the cleaning liquid through the submerged first inlet pipe end 10021 and delivers the cleaning liquid to the ear rinsing assembly 10040, and then the cleaning liquid is expelled through the ear rinsing assembly 10040 into the ear canal.

According to this fourth preferred embodiment of the present invention, the liquid supplying assembly further comprises a gravity ball 190 arranged at the first inlet pipe end 10021 extending into the first cavity of the first liquid storage chamber 10010. When the cleaning liquid is stored within the first cavity 10011A, a first inlet pipe end 10021 of the liquid inlet pipe 10020 is submerged below the liquid level of the cleaning liquid.

The cleaning liquid effectively flushes debris and cerumen from the ear canal, and flows outward carrying the dislodged particles as waste liquid. The waste liquid flows from the ear canal into the liquid collection assembly 20010A, and then flows into the second liquid storage chamber 20030A.

According to this fourth preferred embodiment, the liquid collection assembly 20010A comprises a liquid collection tray 20011, wherein the waste liquid flows out of the ear canal is collected by the liquid collection tray 20011, and then guided into the second liquid storage chamber 20030A. In detail, the liquid collection tray 20011 has at least one flow hole 200111 at a lower portion thereof, allowing the collected waste liquid to flow into the second liquid storage chamber 20030A.

As can be shown in FIG. 14, the liquid collection tray 20011 is designed with a incline to facilitate the smooth flow of waste liquid toward the second liquid storage chamber 20030A. In detail, the liquid collection tray 20011 comprises a guiding plate 200112 that slopes with respect to a horizontal plane H′ defined by the aurilave, wherein the horizontal plane H is vertically aligned with an extension direction L′ defined by the aurilave, wherein with respect to the shell 4000A, the second liquid storage chamber 20030 is installed at a lower position allow the extension direction L, and the first liquid storage chamber 10010 is installed at a higher position.

The liquid collection tray 20011 is installed at an higher position relative to the second liquid storage chamber 20030A, enabling gravitational drainage from the guiding plate 200112 into the second liquid storage chamber 20030A.

The liquid collection tray 20011 further comprises a peripheral flange 200113, wherein the peripheral flange 200113 and the guiding plate 200112 define a collection chamber 200114 that confines the waste liquid and prevents overflow, wherein the peripheral flange 200113 extends upward from the guiding plate 200112 to enclose the lateral boundaries of the collection chamber 200114, ensuring containment prior to gravitational discharge into the temporary storage chamber 200121.

The guiding plate 200112 defines the flow hole 200111 being positioned at a lower elevation while the aurilave being in a usage state, facilitating efficient drainage under gravity.

In detail, in the usage state, the guiding plate 200112 is inclined such that the flow hole 200111 is resided at the lower point, promoting runoff of waste liquid into the second liquid storage chamber 20030A. In more detail, the extending element 10042 of the ear rinsing assembly 10040 is configured to be perpendicular to the guiding plate 200112, and the jet 10043 being installed on the extending element 10042 is perpendicular to the guiding plate 200112. In the usage state, the jet 10043 is inserted into the ear canal at a predetermined angle, ensuring precise delivery of the cleaning liquid. The peripheral flange 200113 surrounds the user's ear and forms a sealed environment to prevent leakage, while the guiding plate 200112 is faced toward the user's ear, forming a protective barrier that prevents splashing, thereby ensuring the waste liquid being efficiently channeled through the flow hole 200111 into the second liquid storage chamber 20030A.

The extending element 10042 is detachably installed to the guiding plate 200112.

The guiding plate 200112 covers the second liquid storage chamber 20030A in such a manner that the flow hole 200111 communicates directly with the second cavity 20031, ensuring a seamless transfer of waste liquid without leakage.

According to this fourth preferred embodiment of the present invention, the liquid outlet pipe 10041 comprises a first outlet pipe 100411 defining a first outlet channel 1004110, a second outlet pipe 100412 defining a second outlet channel 1004120, and a third outlet pipe 100413 defining a third outlet channel 1004130, wherein the first outlet pipe 100411 is connected between the liquid pump 10030 and the second outlet pipe 100412, and the second outlet pipe 100412 is connected between the first outlet pipe 100411 and the third outlet pipe 100413, forming a cascaded fluid pathway, so that the liquid can be drove into the first outlet channel 1004110 and then drove into the second outlet channel 1004120, and subsequently into the third outlet channel 1004130, and then passed through an extending channel 100420 defined by the extension member 10042, which fluidly communicates with the third outlet channel 1004130, thereby guiding the liquid toward the jet 10043 and driving the liquid to be jetted in the ear canal.

The first outlet channel 1004110, the second outlet channel 1004120 communicating with the first outlet channel 1004110, and the third outlet channel 1004130 communicating with the second outlet channel 1004120 form a sequentially aligned liquid outlet channel 100410 that ensures the liquid from the liquid pump 10030 is smoothly and continuously delivered into the extending channel 100420.

The display module 3003 display the signal that indicates the power level of the battery 80. The control unit 3002 is electrically connected to the liquid pump 10030, so as to control the start, the stop, and the flow rate of the liquid pump 10030.

The aurilave further comprises a lighting system 6000. According to this fourth preferred embodiment of the present invention, the lighting system 6000 comprises a LED lamp plate 180 electrically connected to the battery 80 and the control unit 3002, wherein the battery 80 applies power to the LED lamp plate 180. The working states of the LED lamp plate 180 is controlled by the control unit 3002.

In detail, the control system 3000A further comprises a temperature sensor 3004 being extending into the first cavity 10011, so as to sense the temperature of the cleaning liquid in the first cavity 10011. When the temperature sensor 3004 senses the temperature is lower than a first predetermined threshold, the control unit 3002 controls the LED lamp plate 180 to emit a light with a first color, so as to indicate that the cleaning liquid is too cold for optimal use. Conversely, when the temperature exceeds a second predetermined threshold, the control unit 3002 adjusts the LED lamp plate 180 to emit a light with a second color, signaling that the cleaning liquid is too hot. When the temperature is within an acceptable range between the first and second thresholds, the control unit 3002 adjusts the LED lamp plate 180 to emit a light with a third color, indicating that the cleaning liquid is at an ideal temperature for use. This visual feedback

According to this fourth preferred embodiment of the present invention, the first, second, and third colors are red, blue, and green respectively.

Preferably, the first predetermined threshold is 36˜37° C., and the second predetermined threshold is 42˜43° C. According to this fourth preferred embodiment of the present invention, the first predetermined threshold is 37° C., and the second predetermined threshold is 42° C.

The lighting system 6000 further comprises a holder 6001 securing the LED lamp plate 180 in position, and a circuit board 6002 electrically connected to the LED lamp plate 180 and the control unit 3002, enabling signal transmission for real-time temperature indication.

The second outlet pipe 100412 is integrally formed with the holder 6001, wherein the holder 6001 is installed on a top wall 40012 of the shell body 40010, wherein the liquid outlet pipe 10041 further comprises a sealing element 100414 that communicates the first outlet pipe 100411 and the second outlet pipe 100412 is such a manner that a second through hole 400121 formed to the top wall 40012 is seamlessly engaged with the sealing element 100414, ensuring leak-proof connectivity between the first outlet pipe 100411 and the second outlet pipe 100412.

The third outlet pipe 100413 is integrally formed with the second liquid storage chamber 20030A. The second liquid storage chamber 20030A is hermetically connected to shell body 40010 in such a manner that the third outlet pipe 100413 is hermetically engaged with the second outlet pipe 100412, forming a sealed channel for fluid transmission.

The shell body 40010 comprises the top wall 40012 and a surrounding wall 40013 that surrounds the top wall 40012, wherein the surrounding wall 40013 together with the top wall 40012 defines a top cavity 40031 formed to the top portion 40030 and the shell space 40011.

The lighting system 6000 is installed within the top cavity 40031, wherein the second liquid storage chamber 20030A covers the top cavity 40031, and holds the lighting system 6000 in the top cavity 40031.

The second liquid storage chamber 20030A is made of transparent material to allow light emitted from the lighting system 6000 to pass through, so that the colors of the light can be easily observed from the outside.

The guiding plate 200112 can also be made of transparent material to allow light emitted from the lighting system 6000 to pass through, so as to improve brightness in the ear canal and facilitate the user to understand a cleaning situation in the ear canal.

According to other embodiments of the present invention, the aurilave can further comprises a drying assembly, so as to dry the ear canal after cleaning.

According to other embodiments of the present invention, the aurilave can further comprise a heating system electrically connected to the control system 3000A, so as to heat the cleaning liquid in the first cavity 10011A.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and are subject to change without departure from such principles. Therefore, this invention comprises all modifications encompassed within the spirit and scope of the following claims.

What is claimed is:

1. An aurilave for cleaning at least one ear canal, comprising:
a liquid supply assembly supplying cleaning liquid to the ear canal, wherein after cleaning the ear canal, the cleaning liquid is flowed out of the ear canal as waste liquid; and
a waste liquid receiving assembly receiving the waste liquid from the ear canal.

2. The aurilave, as recited in claim 1, wherein said liquid supply assembly comprises a first liquid storage chamber, a liquid inlet pipe, a liquid pump, and an ear rinsing assembly, wherein said first liquid storage chamber defines a first cavity for storing the cleaning liquid, wherein said liquid inlet pipe communicates said first cavity with said liquid pump, enabling said liquid pump to draw the cleaning liquid from said first cavity and deliver the cleaning liquid to said ear rinsing.

3. The aurilave, as recited in claim 2, wherein said waste liquid receiving assembly comprises a second liquid storage chamber defining a second cavity for storing the waste liquid from the ear canal.

4. The aurilave, as recited in claim 3, wherein said waste liquid receiving assembly comprises a liquid collection assembly and a second liquid storage chamber defining a second cavity for storing the waste liquid, wherein said liquid collection assembly is configured to collect the waste liquid from the ear canal, wherein the waste liquid collected by said liquid collection assembly is directed into said second cavity.

5. The aurilave, as recited in claim 4, wherein said first liquid storage chamber comprises a first chamber body and a separating wall, wherein said first chamber body and said separating wall cooperate to define said first cavity, wherein said second liquid storage chamber comprises a second chamber body, wherein said second chamber body and said separating wall cooperate to define said second cavity.

6. The aurilave, as recited in claim 5, wherein said first chamber body, said second chamber body, and said separating wall are formed integrally as a jar.

7. The aurilave, as recited in claim 6, further comprises a shell, wherein said liquid pump is installed into said shell, wherein said jar is detachably installed to said shell.

8. The aurilave, as recited in claim 7, wherein said shell comprises a shell body and a lower cover mounted to said shell body, wherein said lower cover is detachably installed to said jar, so as to hermetically seal said first cavity and said second cavity, ensuring said first cavity and said second cavity remain isolated with each other.

9. The aurilave, as recited in claim 8, said lower cover comprises a second cover body and a sealing strip, wherein said second cover body defines a sealing groove, wherein said sealing strip is disposed within said sealing groove, wherein said sealing strip is elastically compressible to ensure a tight seal between said second cover body and said separating wall.

10. The aurilave, as recited in claim 9, wherein said liquid collection assembly comprises a liquid collection tray and a temporary storage chamber body, wherein the waste liquid flowed from the ear canal is collected in said liquid collection tray and then guided into said temporary storage chamber body, and then directed through said liquid reflow pipe to said second cavity.

11. The aurilave, as recited in claim 10, wherein said liquid collection tray comprises a guiding plate and a peripheral flange, said guiding plate being inclined to facilitate directional flow of waste liquid toward said temporary storage chamber body, wherein said peripheral flange extends upwardly from said guiding plate, wherein said guiding plate defines a flow hole in a lower position.

12. The aurilave, as recited in claim 11, wherein a tilt angle of said guiding plate ranges from 20° to 60°.

13. The aurilave, as recited in claim 12, wherein said ear rinsing assembly comprises a liquid outlet pipe, an extending element, and a jet, wherein said liquid outlet pipe communicate said liquid pump to said extending element, wherein said jet is installed to said extending element, wherein said guiding plate further defines a first through hole allows said extending element to pass through.

14. The aurilave, as recited in claim 13, further comprises a control system, wherein said liquid pump is controlled by said control system, wherein said control system comprises a power supply module, a main control unit, and a display module, wherein said control unit and said display module are electrically connected to said power supply module, wherein said display module display a power level of said power supply module.

15. The aurilave, as recited in claim 4, further comprises a shell, wherein said liquid pump is installed into said shell, wherein with respect to said shell, said first liquid storage chamber and said second liquid storage chamber are installed opposite to each other.

16. The aurilave, as recited in claim 15, wherein said shell has a top portion and a bottom portion, wherein said first liquid storage chamber is installed to said bottom portion, wherein said second liquid storage chamber is installed to said top portion.

17. The aurilave, as recited in claim 16, wherein said wherein said liquid collection assembly comprises a liquid collection tray and a temporary storage chamber body, wherein the waste liquid flowed from the ear canal is collected in said liquid collection tray and then guided into said temporary storage chamber body, and then directed through said liquid reflow pipe to said second cavity.

18. The aurilave, as recited in claim 17, wherein said liquid collection tray comprises a guiding plate and a peripheral flange, said guiding plate being inclined to facilitate directional flow of waste liquid toward said temporary storage chamber body, wherein said peripheral flange extends upwardly from said guiding plate, wherein said guiding plate defines a flow hole in a lower position.

19. The aurilave, as recited in claim 18, wherein a tilt angle of said guiding plate ranges from 20° to 60°.

20. The aurilave, as recited in claim 19, wherein said ear rinsing assembly comprises a liquid outlet pipe, an extending element, and a jet, wherein said liquid outlet pipe communicate said liquid pump to said extending element, wherein said jet is installed to said extending element.

21. The aurilave, as recited in claim 20, further comprises a control system, wherein said liquid pump is controlled by said control system, wherein said control system comprises a power supply module, a main control unit, and a display module, wherein said control unit and said display module are electrically connected to said power supply module, wherein said display module display a power level of said power supply module.

22. The aurilave, as recited in claim 21, further comprises a lighting system electrically connected with said power supply module of said control system.

23. The aurilave, as recited in claim 22, wherein said control system further comprises a temperature sensor extended into said first liquid storage chamber, whereby sensing the temperature of the cleaning liquid in said first liquid storage chamber.

24. The aurilave, as recited in claim 23, wherein said temperature sensor is electrically connected to said main control unit, wherein said main control unit controls colors of light emitted by said lighting system according to the temperature sensed by said temperature sensor.

25. The aurilave, as recited in claim 24, wherein said lighting system is installed to said top portion of said shell, wherein said second liquid storage chamber is made of transparent material.

26. The aurilave, as recited in claim 25, wherein said guiding plate is made of transparent material.
